(19) United States
(12) Patent Application Publication
(10) Pub. No.: US 2017/0296382 A1
(43) Pub. Date: Oct. 19, 2017

(54) MICRO FORCEPS

(71) Applicant: KANEKA CORPORATION, Osaka-shi, Osaka (JP)

(72) Inventor: Yuki MUKAI, Okaya-shi (JP)

(73) Assignee: KANEKA CORPORATION, Osaka-shi, Osaka (JP)

(21) Appl. No.: 15/516,753

(22) PCT Filed: Oct. 2, 2015
(86) PCT No.: PCT/JP2015/078033

§ 371 Date: Apr. 4, 2017

(30) Foreign Application Priority Data
Oct. 24, 2014 (JP) 2014-217254

(51) Int. Cl. A61F 9/007; A61B 17/28

(52) U.S. Cl. CPC A61F 9/007; A61B 17/2841; A61B 17/282; A61B 17/2833

(57) ABSTRACT

A micro forceps 12 according to the present invention comprises: a housing 22 having an opening part 27 on a side 23 thereof; a rotating body 32 that is retained in the housing 22, rotatable around a long axis of the housing 22, and partially exposed from the opening part 27; a clip part 52 for clipping an object; a wire rod part provided with the clip part 52 on one side, and connected to the rotating body 32 on the other side; an opening/closing operation part 62 that is retained in the housing 22, movable along the long axis direction of the housing 22, and partially exposed from the opening part 27; and a tubular part 72 connected to the opening/closing operation part 62; wherein the clip part 52 is in a closed state where the clip part 52 is placed in the tubular part 72 when the opening/closing operation part 62 is moved to a clip part 52 side; and the clip part 52 is in an opened state where the clip part 52 comes out of the tubular part 72 when the opening/closing operation part 62 is moved to opposite to the clip part 52 side.

### TECHNICAL FIELD

The present invention relates to a small medical instrument which is used in surgery of a patient and is called a micro forceps, and relates to a medical instrument which is inserted into an eye of a patient in performing physical medical treatment in an eye, for example.

### BACKGROUND ART

A vitreous body existing in an eye, which has functions of refracting light which is incident upon an eye and maintaining a shape of an eye, occasionally pulls a retina or becomes opaque due to bleeding or the like, to hinder light from arriving at a retina. Vitreous body surgery, in which opaqueness together with a vitreous body is resected or cell membranes proliferating on a retina are removed, is performed in order to restore functions of a retina. A thickness of each of cell membranes proliferating on a retina, for example, is approximately several tens of μm, so that an extremely fine device called a micro forceps (vitreous body forceps) which can clip an object on the order of micrometers is used in vitreous body surgery, and also an operator who uses such a device is required to have high precision in his manipulation.

In order to clip a thin cell membrane or the like, a clip part shaped like scissors is formed at a tip of a micro forceps. An opening/closing operation of the clip part is performed in such a manner that a tubular body placed around a wire rod part having the clip part at a tip of the part is moved along an axis direction of the wire rod part, and scissors are caused to be exposed from the tubular body or be housed into the tubular body. Since a micro forceps is such a fine device as described, it is important to easily achieve alignment of a clipping direction with a clipped object and movement of a tubular body along an axis direction.

For example, Patent literature 1 describes a forceps including a drive clip part formed of an elastic member. In the forceps described in Patent literature 1, a drive device extends when a handle of the forceps is grasped, so that a tip part of the drive clip part is opened/closed. Also, the forceps can clip an object in a whole circumferential direction.

Patent literature 2 describes a micro surgical instrument in which a drive handle including an elastic arm is compressed and pushed inward in a radial direction, so that the elastic arm is bent inward and outward and a ring which is shaped like a handle rod reciprocates, and then a tubular body and a shaft move along an axis direction.

### CITATION LIST

### Patent Literature

### Patent Literature 1

U.S. Pat. No. 6,488,695

### Patent Literature 2

U.S. Patent Application Publication No. 2012/0116361

### SUMMARY OF INVENTION

### Technical Problem

However, the forcipes described in Patent literatures 1 and 2 have a problem of displacement of a clipping position or deviation of a clipping direction during operation.

More specifically, in the forceps described in each of the Patent literatures 1 and 2, in order to adjust a clipping direction through rotation of the clip part around a long axis of the forceps, an operator should rotate a housing itself of the forceps while twisting his arm or wrist, which tends to increase an amount of hands movement of an operator. Thus, if an operator is unpracticed, an inside of an eye may probably be damaged by mistake.

In view of the foregoing matters, according to a first viewpoint, it is an object to provide a micro forceps in which a clipping direction of a clip part can be adjusted without rotating a housing itself of the forceps.

Also, in the forceps described in each of Patent literatures 1 and 2, it is necessary to apply equal forces to handles and almost simultaneously push them inward in a radial direction of the handles with a thumb and a forefinger in order to open or close the clip part. Thus, if an operator is unpracticed, an inside of an eye may probably be damaged due to displacement of a position of the clip part or deviation of a clipping direction.

In view of the foregoing matters, according to a second viewpoint, it is an object to provide a micro forceps in which a clip part can be stably opened or closed without a need to apply a force to inner side in a radial direction of a handle.

### Solution to Problem

A micro forceps according to the present invention, having features of both of a first viewpoint and a second viewpoint and is able to achieve the above object, comprises: a housing having an opening part on a side thereof; a rotating body that is retained in the housing, rotatable around a long axis of the housing, and partially exposed from the opening part; a clip part for clipping an object; a wire rod part provided with the clip part on one side, and connected to the rotating body on the other side, an opening/closing operation part that is retained in the housing, movable along the long axis direction of the housing, and partially exposed from the opening part; and a tubular part connected to the opening/closing operation part; wherein the clip part is in a closed state where the clip part is placed in the tubular part when the opening/closing operation part is moved to the clip part side; and the clip part is in an opened state where the clip part comes out of the tubular part when the opening/closing operation part is moved to opposite to the clip part side. In the micro forceps according to the present invention, a clipping direction of the clip part can be aligned with an object through a rotation operation of the rotating body, so that there is no need to rotate the housing itself around a long axis of the housing. As a result of this, it is unnecessary for an operator to twist his arm and wrist, so that a clipping direction can be stably adjusted. Also, in the micro forceps according to the present invention, the opening/closing operation part can be operated with the housing being grasped and fixed, so that an opening/closing operation can be stably performed. In this manner, the micro forceps according to the present invention allows stable operation, and thus can reduce a probability of occurrence of medical accidents such as displacement of a clipping position or misrecognition of a clipped object.

In the micro forceps, it is preferred that the rotating body has a recessed part on the clip part side in the long axis direction of the housing and is engaged with the opening/closing operation part at the recessed part. The rotating body and the opening/closing operation part are fixed while being engaged with each other, so that a rotation axis of the rotating body and a long axis of the tubular part can be aligned with each other. Also, even if the rotating body is pressed inward in a radial direction, it is possible to prevent a smooth rotation operation from being hampered due to entry of a whole of the rotating body into the housing, because the rotating body is hard to dent. The rotating body is hard to dent because the opening/closing operation part is positioned in an inner portion of a rotation operation part and serves to hold the rotating body.

The above-described micro forceps is used in such a manner that the tubular part is inserted into a lumen of a torquer which is inserted into a body during surgery and serves as an inlet of a body for a surgical equipment such as a forceps. Thus, to insert the tubular part of the micro forceps into the lumen of the torquer having an inner diameter of 1.1 mm or smaller, an outer diameter of the tubular part is preferably 1.1 mm or smaller.

The micro forceps preferably comprises a fixing means that fixes a rotating position of the rotating body. A rotation position of the rotating body can be fixed, so that the rotating body can be prevented from being operated against intention of an operator.

In the micro forceps, the wire rod part preferably has a solid shape or a hollow shape in cross-section. The above-described wire rod part is easy to manufacture, so that industrial mass-production thereof is practicable.

In the micro forceps, the rotating body is preferably engaged with the housing on an opposite side of the clip part side in the long axis direction of the housing. When the rotating body is engaged with the housing, a long axis direction of the housing can be prevented from deviating from rotation axis directions of the rotating body and the wire rod part.

In the micro forceps, the housing and the opening/closing operation part are preferably connected with each other by an elastic member. Either an opening operation or a closing operation of the clip part can be performed by virtue of a restoring force of the elastic member, so that an opening/closing operation of the clip part can be easily performed.

In the micro forceps, the elastic member is preferably a coil spring. Coil springs can be industrially mass-produced, and also, a coil spring can be easily set in a micro forceps.

In the micro forceps, the opening/closing operation part preferably has a finger pad piece projecting in a direction away from the long axis of the housing. When the finger pad piece projects, an operator can easily locate the opening/closing operation part, so that there is no need to confirm a position of the opening/closing operation part of the housing by visual check.

In the micro forceps, an outer end of the finger pad piece is preferably placed at an inner position of an outer end of the opening part. The finger pad piece does not project from the housing, so that the housing does not become hard to grasp even if a finger is in contact with the neighborhood of the opening part all the time during manipulation.

In the micro forceps, an opening range of the opening part in a circumferential direction of the housing is preferably 60° or larger and 180° or smaller relative to the circumferential direction of the housing. As a result of the opening part being provided in the above-described manner, it is possible to stably grasp the housing without degrading operability of the rotating body.

In the micro forceps, the rotating body and the opening/closing operation part are preferably exposed from the opening part. This makes it easy to operate the rotating body and the opening/closing operation part with the same finger, to promote an increase in efficiency in a clipping operation.

In the micro forceps, it is preferred that two of the opening part are formed, the rotating body is exposed from one of the opening part, and the opening/closing operation part is exposed from the other of the opening part. This makes it easy to operate the rotating body and the opening/closing operation part with different fingers, respectively.

### Advantageous Effects of Invention

The micro forceps according to the present invention can reduce a probability of occurrence of medical accidents such as displacement of a clipping position, a deviation of a clipping direction, or misrecognition of a clipped object.

In particular, in the micro forceps according to the present invention based on the first viewpoint, a clipping direction of the clip part can be aligned with an object through a rotation of the rotating body, so that there is no need to rotate the housing itself around a long axis of the housing, and it is unnecessary for an operator to twist his arm and wrist.

Also, in the micro forceps according to the present invention based on the second viewpoint, an opening/closing operation of the clip part can be performed with housing being stably grasped.

### BRIEF DESCRIPTION OF THE DRAWINGS

FIG. 1 is a perspective view of an micro forceps according to a first embodiment.

FIG. 2 is a cross-sectional view (a partial side view) taken along an axis direction of the micro forceps according to the first embodiment.

FIG. 3 is an assembly drawing of a tip part of the micro forceps in FIG. 1.

FIG. 4 is a perspective view showing a connection between a rotating body and a wire rod part according to the first embodiment.

FIG. 5 is a cross-sectional view (a partial side view) taken along an axis direction of the micro forceps according to the first embodiment.

FIG. 6 is a perspective view of an micro forceps according to a second embodiment.

FIG. 7 is a perspective view showing a connection between an opening/closing operation part and a tubular part according to the second embodiment.

FIG. 8 is a cross-sectional view (a partial side view) taken along an axis direction of the micro forceps according to the second embodiment.

FIG. 9 is a perspective view of an micro forceps according to a third embodiment.

FIG. 10 is a perspective view showing a connection between a rotating body and a wire rod part according to the third embodiment.

FIG. 11 is a perspective view showing a connection between an opening/closing operation part and a tubular part according to the third embodiment.

FIG. 12 is a cross-sectional view (a partial side view) taken along an axis direction of the micro forceps according to the third embodiment.

FIG. 13 is a perspective view showing an engagement between an opening/closing operation part and a rotating body according to the third embodiment.

FIG. 14 is a side view of the opening/closing operation part according to the third embodiment.

FIG. 15 is a front view of the rotating body according to the third embodiment.

### DESCRIPTION OF EMBODIMENTS

(A First Embodiment Based on a First Viewpoint)

An micro forceps provided with a rotating body according to first embodiment comprises: a housing having an opening part on a side thereof; a rotating body that is retained in the housing, rotatable around a long axis of the housing, and partially exposed from the opening part; a clip part for clipping an object; and a wire rod part provided with the clip part on one side, and connected to the rotating body on the other side. In the micro forceps provided with the rotating body according to the present invention, a clipping direction of the clip part can be aligned with an object through a rotation operation of the rotating body, so that there is no need to rotate the housing itself around a long axis of the housing. Thus can reduce a probability of occurrence of medical accidents such as displacement of a clipping position or misrecognition of a clipped object.

(A Second Embodiment Based on a Second Viewpoint)

Moreover, an micro forceps provided with an opening/closing operation part which is movable along the long axis direction of the housing according to the second embodiment comprises: a housing having an opening part on a side thereof; an opening/closing operation part that is retained in the housing, movable along the long axis direction of the housing, and partially exposed from the opening part; a tubular part connected to the opening/closing operation part; a clip part for clipping an object; a wire rod part placed in a lumen of the tubular part, and provided with the clip part on one side; wherein the clip part is in a closed state where the clip part is placed in the tubular part when the opening/closing operation part is moved to the clip part side; and the clip part is in an opened state where the clip part comes out of the tubular part when the opening/closing operation part is moved to opposite to the clip part side. In the micro forceps provided with an opening/closing operation part which is movable along the long axis direction, the opening/closing operation can be performed with the housing being stably grasped, and thus can reduce a probability of occurrence of medical accidents such as displacement of a clipping position or misrecognition of a clipped object.

(A Third Embodiment Having Features of Both of the First Viewpoint and the Second Viewpoint)

An micro forceps having features of both of the first viewpoint and the second viewpoint according to the present invention comprises: a housing having an opening part on a side thereof; a rotating body that is retained in the housing, rotatable around a long axis of the housing, and partially exposed from the opening part; a clip part for clipping an object; a wire rod part provided with the clip part on one side, and connected to the rotating body on the other side; an opening/closing operation part that is retained in the housing, movable along the long axis direction of the housing, and partially exposed from the opening part; and a tubular part connected to the opening/closing operation part; wherein the clip part is in a closed state where the clip part is placed in the tubular part when the opening/closing operation part is moved to the clip part side; and the clip part is in an opened state where the clip part comes out of the tubular part when the opening/closing operation part is moved to opposite to the clip part side. In the micro forceps according to the present invention, a clipping direction of the clip part can be aligned with an object through a rotation operation of the rotating body, so that there is no need to rotate the housing itself around a long axis of the housing. As a result of this, it is unnecessary for an operator to twist his arm and wrist, so that a clipping direction can be stably adjusted. Also, in the micro forceps according to the present invention, the opening/closing operation part can be operated with the housing being grasped and fixed, so that an opening/closing operation can be stably performed. As described above, the micro forceps according to the present invention allows stable operation, and thus can reduce a probability of occurrence of medical accidents such as displacement of a clipping position or misrecognition of a clipped object.

Generally, a micro forceps is inserted into a lumen of a tubular body called a torquer which has a size between 27 G and 19 G inclusive (an inner diameter is 0.4 mm or larger and 1.1 mm or smaller), for use. For example, in vitreous body surgery, a plurality of torquers are disposed on an eye ball, and a luminaire, a reflexed-liquid infusing device, and the like, in addition to a micro forceps, are inserted into holes (lumens) of the torquers. In the present invention, “G (gauge)” represents a size in accordance with Birmingham Wire Gauge.

A way to hold a micro forceps varies from operator to operator. For example, an operator grasps a micro forceps with his three fingers of a thumb, a forefinger, and a middle finger as if he holds a pencil, and another operator grasps a clip part side of a housing with his four fingers of a thumb, a forefinger, a middle finger, and a ring finger as if he holds a writing brush. In both ways, it is general to hold any position from a central to a hind part of a housing with a web part between a thumb and a forefinger.

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

In the present invention, an axis direction of a micro forceps refers to a long axis direction of a housing. Also, in a long axis direction, a front with respect to a micro forceps refers to a direction of a clip-part side, and a rear with respect to a micro forceps refers to a direction opposite to a clip part of a housing. A radial direction of a micro forceps refers to a radial direction of a housing, and being inward in a radial direction refers to a direction toward a center of a housing, while being outward refers to a radiation direction of a housing.

A first embodiment corresponding to the above-described first viewpoint will describe a micro forceps provided with a rotating body, a second embodiment corresponding to the above-described second viewpoint will describe a micro forceps provided with an opening/closing operation part movable along a long axis direction of a housing, and a third embodiment having respective features of the above-described first and second viewpoints will describe a micro forceps provided with both of a rotating body and an opening/closing operation part.

### A First Embodiment

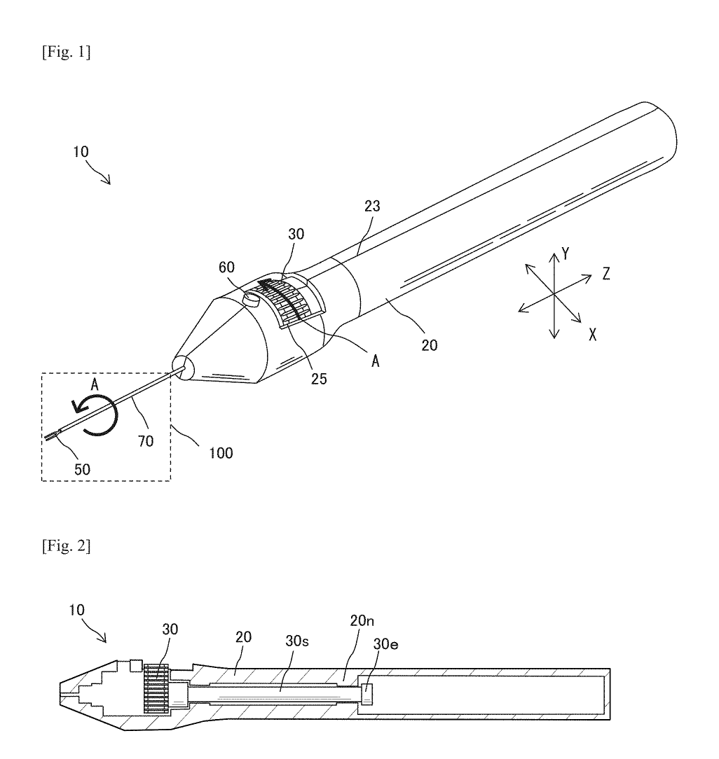

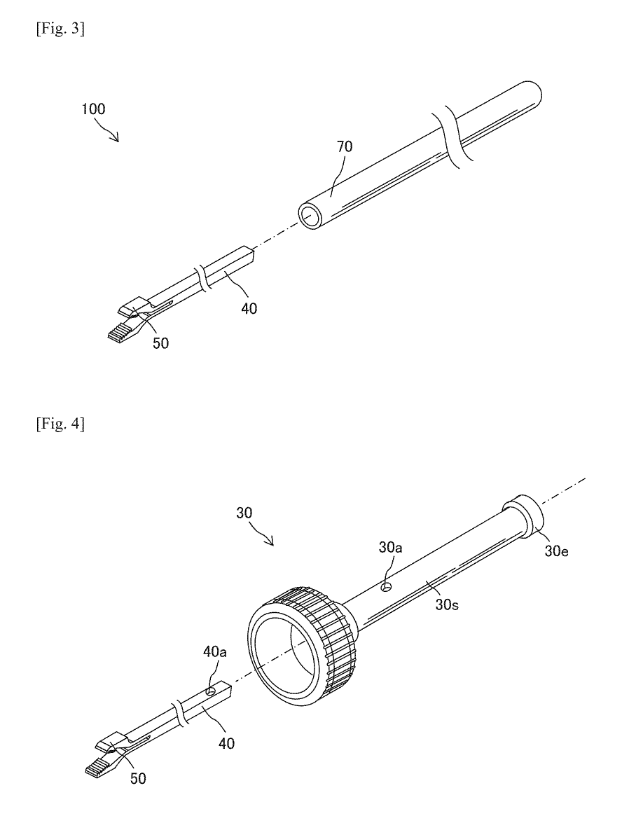

FIG. 1 is a perspective view of an micro forceps 10 according to a first embodiment, FIG. 2 is a cross-sectional view (a partial side view) taken along an axis direction of the micro forceps 10 according to the first embodiment, and FIG. 3 is an assembly drawing of a tip part 100 of the micro forceps 10 in FIG. 1. The micro forceps 10 according to the present invention comprises a housing 20, a rotating body 30 in the housing 20, and a wire rod part 40 in the housing 20. The housing 20 having an opening part 25 on a side 23 and a rotating body 30 that is retained in the housing 20. An operator operates the micro forceps 10 by grasping the side 23 of the housing 20.

More specially, the rotating body 30 is rotatable around a circumferential direction of the housing 20, retained in a long axis direction (Z direction in FIG. 1) of the housing 20, and partially exposed from the opening part 25. Also, the rotating body 30 is connected to the wire rod part 40. Accordingly, when a tangential force is applied to the rotating body 30 which is exposed from the opening part 25 of the housing 20 with a finger being brought in contact with the rotating body 30, the rotating body 30 rotates in A direction as shown in FIG. 1, for example, and also the wire rod part 40 connected to the rotating body 30 and a clip part 50 rotate in A direction.

It is preferred that the housing 20 has a size and a shape which are suitable for an operator to operate the housing 20 while grasping the housing 20 with one hand. For example, it is preferred that the housing 20 has a circular tubular shape, a quadrangular tubular shape, a pentagonal tubular shape, a hexagonal tubular shape, or a polygonal tubular shape having more apexes or sides. Also, a tip part of the housing 20 is preferably tapered toward a tip thereof in order to reduce blind spots as seen by an operator. Also, it is preferable that the housing 20 has a size which is small enough for an operator to grasp with one hand, and the housing 20 can be designed so as to have a length between approximately 8 cm and 25 cm inclusive, and a maximum diameter between approximately 1 cm and 5 cm inclusive, a minimum diameter between approximately 0.5 cm and 4 cm inclusive, for example, like a writing instrument, a tooth brush, or an electric knife. Further, in order to make it easy to grasp the housing 20, unevenness or a non-slip member having a high friction coefficient, such as rubber or a synthetic resin, may be provided in the side 23 of the housing 20.

A shape of the opening part 25 is not limited to any particular shape. However, if the opening part 25 is too wide, the rotating body 30 cannot be effectively retained, so that an axis of the rotating body 30 may probably deviate. If the opening part 25 is too narrow, an exposed area of the rotating body 30 is small, so that an operator has difficulties in bringing his finger into contact with the rotating body 30. As such, a suitable shape of the opening part 25 which satisfies two requirements of prevention of axis deviation of the rotating body 30 and reservation of an exposed area is a rectangle as viewed from above the opening part 25, in other words, in plan view, as shown in FIG. 1. On the other hand, a width of the opening part 25 in the long axis direction of the housing 20 can be set so as to decrease in a tapered manner, or increase in a tapered manner, in a rotation direction of the rotating body 30 so that an operator can comprehend an amount of rotation of the rotating body 30 even through feeling at his finger tip.

An opening range of the opening part 25 in a circumferential direction of the housing 20 is preferably 60° or larger and 180° or smaller relative to the circumferential direction of the housing 20. As a result of the opening part 25 being provided in the above-described manner, it is possible to stably grasp the housing 20 without degrading operability of the rotating body 30. In order to secure stability of an axis, an upper limit of the opening range of the opening part 25 in the circumferential direction of the housing 20 is more preferably 170° or smaller, is further preferably 160° or smaller, and is still further preferably 150° or smaller. Meanwhile, if the opening range of the opening part 25 in the circumferential direction of the housing 20 is too narrow, an operator should repeatedly reciprocate his finger in a circumferential direction of the opening part 25 in order to rotate the rotating body 30, which reduces operability. Thus, a lower limit of the opening range of the opening part 25 in the circumferential direction of the housing 20 is more preferably 70° or larger, is further preferably 80° or larger, and is still further preferably 90° or larger. It is additionally noted that, in setting a numerical range, the above-stated values of upper limits and lower limits of an opening range can be arbitrarily combined. This holds true for any other limitations to a numerical range in the present specification.

In order to prevent the rotating body 30 from being operated against intention of an operator, the micro forceps 10 preferably comprises a fixing means that fixes a rotating position of the rotating body 30. Though not shown in the drawings, a plurality of grooves can be provided in an outer side surface of the rotating body 30 along the long axis direction of the housing 20, as the fixing means, for example. By providing projections which are engaged with those grooves on an inner side surface of the housing 20, it is possible to fix a rotating position of the rotating body 30 in such a manner that a whole of the rotating body 30 is moved to a forepart of the housing 20 after the rotating body 30 is stopped at a desired position, and the grooves serving as the fixing means and the projections on an inner side surface of the housing 20 are brought into engagement with each other.

Alternatively, an engagement means which is engaged with the housing 20 can be provided in a hind part of the rotating body 30. That is, it is preferred that the rotating body 30 is engaged with the housing 20 in a hind part in the long axis direction of the housing 20. As shown in FIG. 2, the rotating body 30 is provided with a shaft part 30s, and a large diameter part 30e having an outer diameter which is larger than that of the shaft part 30s is provided in the rear of the shaft part 30s. On the other hand, in the housing 20, a small width part 20n having an inner diameter which is larger than that of the shaft part 30s and is smaller than that of the large diameter part 30e is provided in the rear of the shaft part 30s. When the rotating body 30 attempts to move in the long axis direction of the housing 20, the large diameter part 30e is blocked by the small width part 20n. Accordingly, respective positions of the rotating body 30 and the wire rod part 40 in the long axis direction of the housing 20 can be prevented from being displaced.

In a case where the rotating body 30 and the wire rod part 40 are directly fixed to each other, a rotation angle of the rotating body 30 is equal to a rotation angle of the wire rod part 40. However, in a case where the rotating body 30 and the wire rod part 40 are connected to each other by a gear, the rotating body 30 is allowed to rotate at a rate higher than the wire rod part 40. Then, it is possible to widely rotate the clip part 50 while lowering a moving range of an operator's finger and reducing hands movement.

A contour of a section orthogonal to a long axis direction of the wire rod part 40 can be designed so as to have a shape of a circle, an ellipse, or a rectangle, for example. Also, the wire rod part 40 preferably has a solid shape in cross-section. The wire rod part 40 which has solid shape in cross-section is easy to manufacture, and industrial mass production thereof is practicable, so that a manufacturing cost can be reduced. On the other hand, in a case where the wire rod part 40 is formed so as to be hollow, flexibility can be improved. In this case, a proportion of an area of a hollow part to a whole section of the wire rod part 40 is between 20% and 80% inclusive, and is preferably between 30% and 70% inclusive, and is more preferably between 40% and 60% inclusive.

As shown in FIG. 3, the wire rod part 40 is placed in a lumen of the tubular part 70 to be described later. In order to allow the wire rod part 40 to smoothly move in a long axis direction of the tubular part 70, a maximum diameter of the wire rod part 40 is set to be not more than 90% of a minimum inner diameter of the tubular part 70, is preferably set to be not more than 80%, and is more preferably set to be not more than 70%. Also, in a long axis direction, the wire rod part 40 is only required to have a length which is small enough for the housing 20 and the tubular part 70 to house.

While it is preferable that a material of the wire rod part 40 has biocompatibility, the wire rod part 40 used in the present invention is to be under a great load also in a twisting direction, and thus should have a certain degree of strength. Accordingly, a material of the wire rod part 40 is preferably a metallic material, a rigid plastic material, a carbon material, or the like. In a case where a metallic material is used, a stainless steel, a nickel-titanium alloy, or a cobalt-nickel alloy can be used, for example. For use of a stainless steel, it is preferred that an amount of carbon is lowered in order to prevent the wire rod part 40 from being brittle.

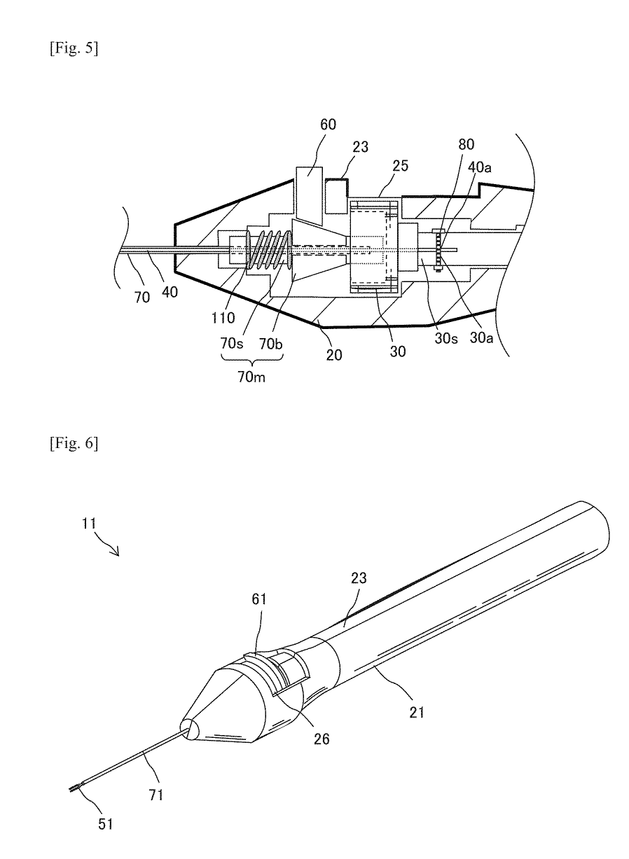

FIG. 4 is a perspective view showing a connection between the rotating body 30 and the wire rod part 40 according to the first embodiment, and FIG. 5 is a cross-sectional view (a partial side view) taken along an axis direction of the micro forceps 10 according to the first embodiment.

The rotating body 30 and the wire rod part 40 are connected to each other in the following manner. For example, as shown in FIG. 4 and FIG. 5, a first through hole 40a penetrating in a direction orthogonal to a long axis direction is provided in a hind part of the wire rod part 40, and a second through hole 30a penetrating in a direction orthogonal to a long axis direction is provided in a forepart of the shaft part 30s. Then, the first through hole 40a and the second through hole 30a are disposed so as to overlap each other. A connecting member 80 such as a bolt is inserted into the shaft part 30s from outside so as to pass through the first through hole 40a and the second through hole 30a, and a tip of the connecting member 80 which is exposed outward from the shaft part 30s is fastened by a nut, so that the rotating body 30 and the wire rod part 40 are connected to each other.

The rotating body 30 and the wire rod part 40 can be connected to each other by the other methods such as laser welding, solder welding, brazing, bonding, swaging, or screwing, for example, in addition to the above-described method using a bolt and a nut. In this regard, it is preferred that the rotating body 30 and the wire rod part 40 are connected in such a manner that with respective axial centers of the rotating body 30 and the wire rod part 40 overlap each other in order to prevent deviation of an axis of the rotating body 30.

The clip part 50 can be used for picking up a clipped object (cell membranes proliferating on a retina, for example) and removing the clipped object from an eye of a patient, and is provided at one end of the wire rod part 40 as shown in FIG. 3. That is, since the clip part 50 is connected to the rotating body 30 via the wire rod part 40, the clip part 50 rotates along with rotation of the rotating body 30 as described above. As such, by rotating the rotating body 30, it is possible to adjust a direction in which the clip part 50 clips an object or an angle at which the clip part 50 clips an object. In other words, in the present invention, it is possible to adjust a clipping direction of the clip part 50 without rotating the housing 20 itself.

The clip part 50 can be formed into an arbitrary shape in accordance with a request of an operator who performs vitreous body surgery, and can be formed as a clip part having a shape of tips of tweezers, for example.

A material of the clip part 50 preferably has biocompatibility as well as the wire rod part 40, and can be used a stainless steel, a nickel-titanium alloy, or a cobalt-nickel alloy can be used, for example.

The clip part 50 and the wire rod part 40 may be formed integrally with each other from the same material, or may be connected to each other after the clip part 50 and the wire rod part 40 are formed as individual members, respectively. While a method of connecting the clip part 50 and the wire rod part 40 is not limited to any particular method, laser welding, solder welding, or swaging can be used, for example.

Also, the present invention includes the micro forceps 10 in which the tubular part 70 is provided around the wire rod part 40. In the tubular part 70, a lumen houses the wire rod part 40 and the clip part 50 so as not to damage body tissue which is a non-clipped object. As shown in FIG. 1 and FIG. 5, an opening/closing operation part 60 which can move along a radial direction and has a shape of a diagonally-cut rod is provided in the side 23 of the housing 20 in the micro forceps 10. Around the tubular part 70, a conical member 70m including a flange part 70b which is tapered toward a hind part thereof is provided so as to extend along the opening/closing operation part 60. Though FIG. 5 shows an example in which a circular tubular body provided with the flange part 70b around a part of the cylinder is provided as the conical member 70m, the conical member 70m may be a rectangular tubular body provided with the flange part 70b.

When the opening/closing operation part 60 is pressed inward in a radial direction of the housing 20, the flange part 70b of the conical member 70m is pushed forward relative to the wire rod part 40, and the tubular part 70 moves forward, so that the wire rod part 40 and the clip part 50 are housed in the tubular part 70 and the clip part 50 is in a closed state. On the other hand, when the opening/closing operation part 60 is released from a press, the clip part 50 is exposed from the tubular part 70 and is in an opened state. As such, by pressing the opening/closing operation part 60 and releasing the opening/closing operation part 60 from a press, it is possible to perform an opening/closing operation of the clip part 50.

The micro forceps 10 is used by inserting the tubular part 70 into the lumen of the torquer. Thus, an outer diameter of the tubular part, for example, is preferably 0.4 mm or larger, is more preferably 0.45 mm or larger, is further preferably 0.5 mm or larger, also is preferably 1.1 mm or smaller, is more preferably 1.05 mm or smaller, is further preferably 1.0 mm or smaller.

A material of the tubular part 70 preferably has biocompatibility, and is preferably a metallic material, a rigid plastic material, a carbon material or the like. In a case where a metallic material is used, a stainless steel or the like can be used, and in a case where a plastic material is used, a polycarbonate, an ABS resin or the like can be used.

The tubular part 70 and the conical member 70m may be formed integrally with each other from the same material in a manner similar to the manner in which the clip part 50 and the wire rod part 40 are formed. Otherwise, the tubular part 70 and the conical member 70m may be connected to each other after being formed as individual members, respectively. As a method of connecting the tubular part 70 and the conical member 70m, laser welding, solder welding, or swaging can be used, for example.

Also, as shown in FIG. 5, the housing 20 and the conical member 70m are preferably connected with each other by an elastic member 110. In FIG. 5, the conical member 70m includes a shaft part 70s in which an outer diameter at an end closer to the clip part 50 is smaller than a maximum outer diameter of the flange part 70b, and a coil spring is provided as an elastic member 110 around the shaft part 70s. When the tubular part 70 is moved forward as a result of the opening/closing operation part 60 being pressed with a finger, the flange part 70b of the conical member 70m which is engaged with the opening/closing operation part 60 moves forward, so that the clip part 50 is housed in the tubular part 70 and is in a closed state. Then, the coil spring is compressed in a long axis direction of the housing 20, so that elastic energy is stored. When the opening/closing operation part 60 is released from a press, the coil spring is restored to the same shape as it had before compression, and the conical member 70m returns to the same position as it was before a press in a long axis direction of the housing 20. Thus, the clip part 50 is exposed outward from the tubular part 70, and is in an opened state. In the above-described manner, by connecting the housing 20 and the opening/closing operation part 60 by the elastic member 110, it is possible to easily perform an opening/closing operation of the clip part 50.

Note that the type of the elastic member is not limited, the elastic member is particularly preferably a coil spring. Since a coil spring can be industrially mass-produced, and also it is only necessary to position it around the shaft part 70s of the conical member 70m, a coil spring can be easily set in a micro forceps 10. A shape, a material, a pitch of the coil spring can be appropriately set in accordance with a shape of the opening/closing operation part 60 or the housing 20, an operator's preference regarding sense of use, and the like.

A conventional micro forceps puts a burden on a patient because a tubular part rotates along with rotation of a housing. In contrast, in the micro forceps 10 according to the present embodiment, there is no need to rotate the housing 20 itself around a long axis of the housing 20. Also, in the micro forceps 10 according to the embodiment of the present invention, the rotating body 30 and the tubular part 70 are not connected to each other, so that the tubular part 70 does not rotate even if the rotating body 30 is rotated, which allows less-invasive treatment.

### A Second Embodiment

FIG. 6 is a perspective view of an micro forceps 11 corresponding to the above-described second viewpoint according to a second embodiment of the present invention. Note that a description of the same components as those of the micro forceps 10 of the first embodiment is omitted.

A micro forceps 11 comprises: a housing 21 having an opening part 26 on a side 23; an opening/closing operation part 61 that is retained in the housing 21, movable along the long axis direction of the housing 21, and partially exposed from the opening part 26; a tubular part 71 connected to the opening/closing operation part 61; a clip part 51 for clipping an object; and a wire rod part placed in a lumen of the tubular part 71 and provided with the clip part 51 on one side.

The opening/closing operation part 61 changes a position of the tubular part 71 relative to the clip part 51 in a long axis direction of the housing 21 and thus is the clip part 51 in an opened state or a closed state, in performing a clipping operation on object tissue of a patient. According to the second embodiment, the opening/closing operation part 61 is exposed from the opening part 26 formed in the side 23 of the housing 21. Thus, when a finger is brought into contact with the opening part 26 and the opening/closing operation part 61 being exposed is moved toward the clip part 51, the clip part 51 which is in an opened state is covered with the tubular part 71 from the rear thereof as a result of forward movement of the tubular part 71 relative to the clip part 51. At that time, the clip part 51 becomes gradually smaller in a radial direction along with a shape of an inner side surface of the tubular part 71, and finally, the clip part 51 is housed in the tubular part 71 and is in a closed state. In contrast thereto, when the opening/closing operation part 61 is moved to opposite to the clip part 51 side, in other words, is moved to a hind part of the housing 21, the tubular part 71 moves backward relative to the clip part 51. At that time, the clip part 51 becomes gradually larger in a radial direction along with a shape of an inner side surface of the tubular part 71, and finally, the clip part 51 exits from the tubular part 71 to an outside, and is in an opened state. As such, by operating the opening/closing operation part 61, it is possible to move the tubular part 71 in a long axis direction of the housing 21.

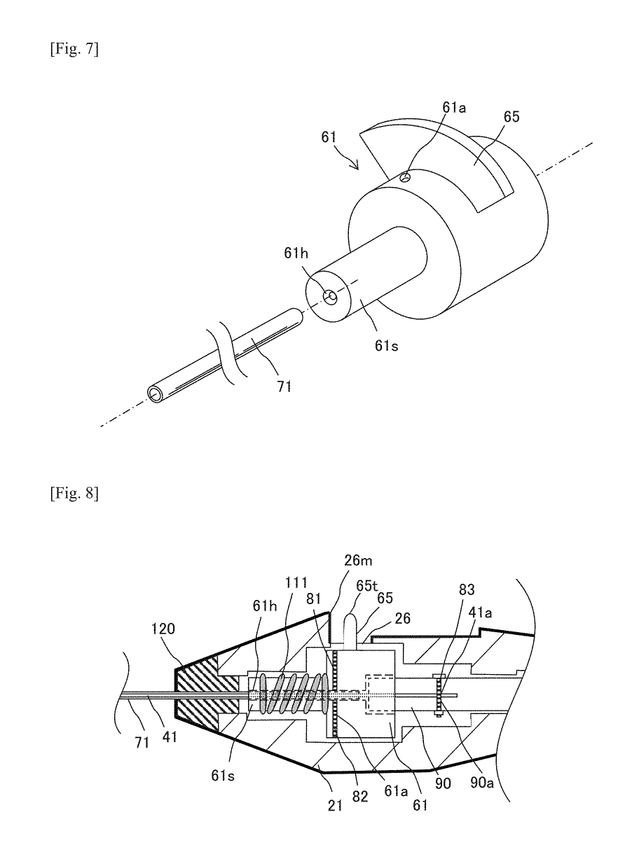

FIG. 7 is a perspective view showing a connection between an opening/closing operation part 61 and a tubular part 71 according to the second embodiment, and FIG. 8 is a cross-sectional view (a partial side view) taken along an axis direction of the micro forceps 11 according to the second embodiment. The opening/closing operation part 61 and the tubular part 71 can be connected to each other in the following manner. A third through hole 61a penetrating in a direction orthogonal to a long axis direction of the housing 21 and a hole part 61h by which at least a part of the tubular part 71 is housed in the opening/closing operation part 61 are provided in the opening/closing operation part 61. A hind part of the tubular part 71 is inserted into the hole part 61h of the opening/closing operation part 61, and connecting members 81 and 82 such as slotted set screws, for example, are inserted into the third through hole 61a. Then, the tubular part 71 is externally tightened, so that the opening/closing operation part 61 and the tubular part 71 are connected to each other.

The opening/closing operation part 61 and the tubular part 71 can be connected to each other by the other methods such as laser welding, solder welding, brazing, bonding, or swaging, for example, in addition to the above-described method using a bolt and a nut. In this regard, it is preferred that the opening/closing operation part 61 and the tubular part 71 are connected in such a manner that with respective axial centers of the opening/closing operation part 61 and the tubular part 71 overlap each other.

In order to retain a hind part of the wire rod part 41 in the housing 21, a holding member 90 may be provided in the hind part of the opening/closing operation part 61. The holding member 90 can be formed of a circular tubular body or a rectangular tubular body which can house the wire rod part 41 in an inside thereof, for example. The wire rod part 41 is connected to the holding member 90 so as not to move in a long axis direction of the housing 21. Accordingly, when the opening/closing operation part 61 is moved forward and backward relative to the wire rod part 41 and the holding member 90 in a long axis direction of the housing 21, the tubular part 71 connected to the opening/closing operation part 61 can be moved forward and backward in a long axis direction, so that an opening/closing operation of the clip part 51 can be performed.

Preferably, when the opening/closing operation part 61 is moved to a front end, a part of the holding member 90 is housed, or fitted and housed, in the hole part 61h of the opening/closing operation part 61. As shown in FIG. 8, by disposing the holding member 90 in such a manner that a part of the holding member 90 is housed in the hole part 61h, it is possible to prevent axis deviation of the holding member 90 in moving the opening/closing operation part 61 forward and backward in a long axis direction of the housing 21. Accordingly, when the opening/closing operation part 61 is moved forward and backward in a long axis direction of the housing 21 and the tubular part 71 is moved forward and backward in a long axis direction, not only forward and backward movement of the wire rod part 41 in a long axis direction, but also axis deviation of the wire rod part 41, can be prevented, so that an opening/closing operation of the clip part 51 can be more stably performed.

The wire rod part 41 and the holding member 90 are connected to each other in the following manner in the same method as the connection between the rotating body 30 and the wire rod part 40 according to the first embodiment. For example, as shown in FIG. 7 and FIG. 8, a first through hole 41a penetrating in a direction orthogonal to a long axis direction is provided in a hind part of the wire rod part 41, and a fourth through hole 90a penetrating in a direction orthogonal to a long axis direction is provided in a forepart of the holding member 90. Then, the first through hole 41a and the fourth through hole 90a are disposed so as to overlap each other. A connecting member 83 such as a bolt is inserted into the holding member 90 from outside so as to pass through the first through hole 41a and the fourth through hole 90a, and a tip of the bolt which is exposed outward from the holding member 90 is fastened by a nut, so that the wire rod part 41 and the holding member 90 are connected to each other.

In the same method as the connection between the opening/closing operation part 61 and the tubular part 71, the wire rod part 41 and the holding member 90 can be connected to each other by the other methods such as laser welding, solder welding, brazing, bonding, swaging, or screwing. In this regard, it is preferred that the wire rod part 40 and the holding member 90 are connected in such a manner that with respective axial centers of the wire rod part 40 and the holding member 90 overlap each other. It is additionally noted that though the holding member 90 is used for retaining a hind part of the wire rod part 41 in the housing 21 in the second embodiment, the wire rod part 41 and the housing 21 can be directly connected to each other in order to reduce the number of composing members.

As shown in FIG. 7 and FIG. 8, the opening/closing operation part 61 preferably has a finger pad piece 65 projecting in a direction away from the long axis of the housing 21, that is, outward in a radial direction. When the finger pad piece 65 projects outward in a radial direction, an operator can easily locate the opening/closing operation part 61, so that there is no need to confirm a position of the opening/closing operation part 61 of the housing 21 by visual check. As a result of this, an opening/closing operation of the clip part 51 can be smoothly performed, so that an operator can efficiently proceed with surgery.

As shown in FIG. 8, an outer end 65t of the finger pad piece 65 is preferably placed at an inner position of an outer end 26m of the opening part 26. The finger pad piece 65 does not project from the housing 21, so that the housing 21 does not become hard to grasp even if a finger is in contact with the outer end 26m of the opening part 26 all the time during manipulation. In this regard, the outer end 26m of the opening part 26 means an end being in the farthest position from an axis center of the opening/closing operation part 61 outward in a radial direction. An outer end 65t of the finger pad piece 65 means an end of the finger pad piece 65, which is in the farthest position from an axis center of the opening/closing operation part 61 outward in a radial direction.

Also, it is preferred that the finger pad piece 65 is hidden from a view by the housing 21 in both of a case where the housing 21 is seen from a the clip part 51 side in a long axis direction and a case where the housing 21 is seen from opposite to the clip part 51 side. Since the finger pad piece 65 does not project from the housing 21, an operator is prevented from having difficulties in grasping even if his finger is in contact with the outer end 26m of the opening part 26 all the time during manipulation.

Also, as shown in FIG. 8, it is preferred that a lumen of the housing 21 has a shape which is engaged with a contour of the opening/closing operation part 61 in order to make it easy to retain the opening/closing operation part 61 in the housing 21. Further, it is preferred that the housing 21 and the opening/closing operation part 61 are connected to each other by an elastic member 111. In FIG. 8, the opening/closing operation part 61 includes a shaft part 61s in which an outer diameter at the clip part 51 side is smaller than an outer diameter at an opening side. A coil spring is provided around the shaft part 61s, as the elastic member 111. When the opening/closing operation part 61 is pressed and moved toward the clip part 51 with a finger and is kept as it is, the clip part 51 is housed in the tubular part 71 and is in a closed state. Then, the coil spring is compressed in a long axis direction of the housing 21, so that elastic energy is stored. When the opening/closing operation part 61 is released from a press, the coil spring is restored to the same shape as it had before compression, and the opening/closing operation part 61 is pushed back to a hind part of the housing 21, so that the clip part 51 is exposed outward from the tubular part 71 and is in an opened state. By connecting the housing 21 and the opening/closing operation part 61 by the elastic member 111 in the above-described manner, it is possible to easily perform an opening/closing operation of the clip part 51.

Note that the type of the elastic member 111 is not limited, the elastic member 111 is particularly preferably a coil spring. Since a coil spring can be industrially mass-produced, and also it is only necessary to position it around the shaft part 61s of the opening/closing operation part 61, a coil spring can be easily set in the micro forceps 11. A shape, a material, a pitch of the coil spring can be appropriately set in accordance with a shape of the opening/closing operation part 61 or the housing 21, an operator's preference regarding sense of use, and the like.

In FIG. 8, while a part of the tubular part 71 is housed in the housing 21, if respective materials forming the tubular part 71 and the housing 21 are different from each other in stiffness, a value of stress on a boundary between the two different materials increases. Thus, there is caused a concern that a possibility of breakage of the micro forceps 11 may increase. Accordingly, it is preferred that a buffering member 120 is provided between the tubular part 71 and the housing 21 as shown in FIG. 8. In particular, it is preferred that the buffering member 120 is provided between a forepart of the housing 21 where stress tends to concentrate and the tubular part 71. It is preferred that the buffering member 120 is elastic, and so the buffering member 120 can be formed of rubber, or a synthetic resin for example.

### A Third Embodiment

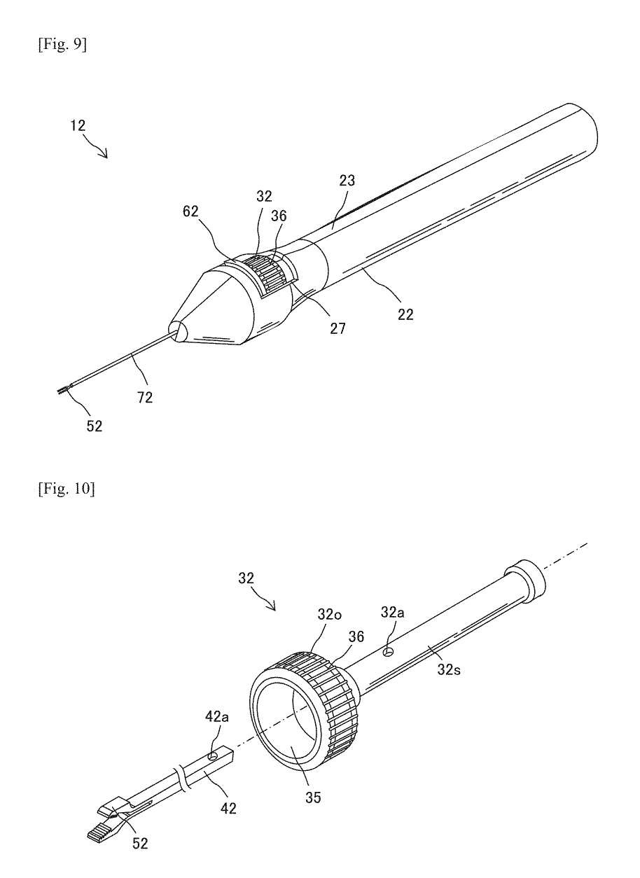

FIG. 9 is a perspective view of an micro forceps 12 having features of both of the first viewpoint and the second viewpoint according to the third embodiment. Note that a description of the same components as those of the micro forceps 10 and 11 of the first and second embodiments is omitted.

The micro forceps 12 comprises: a housing 22 having an opening part 27 on a side 23 thereof; a rotating body 32 that is retained in the housing 22, rotatable around a long axis of the housing 22, and partially exposed from the opening part 27; and an opening/closing operation part 62 that is retained in the housing 22, movable along the long axis direction of the housing 22, and partially exposed from the opening part 27.

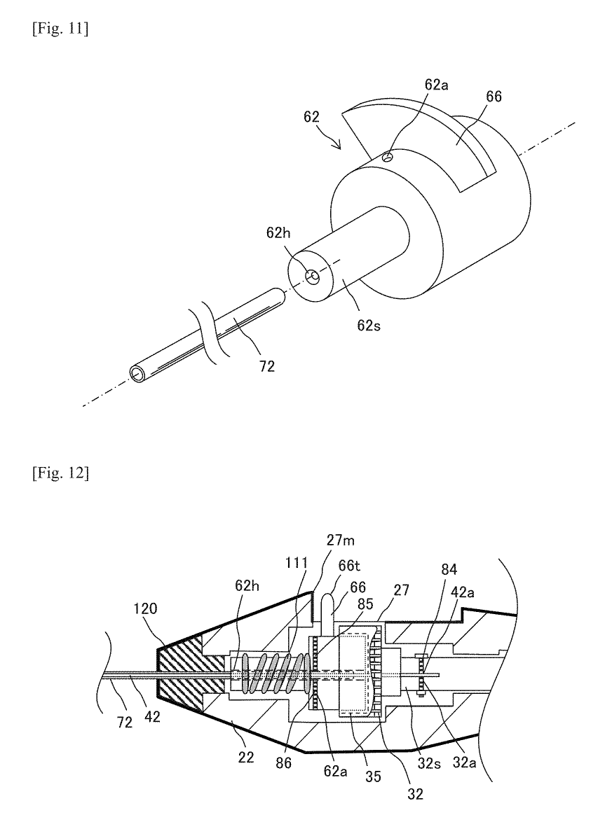

FIG. 10 is a perspective view showing a connection between the rotating body 32 and a wire rod part 42 according to the third embodiment, FIG. 11 is a perspective view showing a connection between an opening/closing operation part 62 and a tubular part 72 according to the third embodiment, and FIG. 12 is a cross-sectional view (a partial side view) taken along an axis direction of the micro forceps 12 according to the third embodiment. The wire rod part 42 is provided with the clip part 52 on one side, and connected to the rotating body 32 on the other side, and the tubular part 72 is connected to the opening/closing operation part 62.

The rotating body 32 and the wire rod part 42 can be connected to each other in the same method as the connection between the rotating body 30 and the wire rod part 40 according to the first embodiment. As shown in FIG. 10 and FIG. 12, for example, a first through hole 42a penetrating in a direction orthogonal to a long axis direction is provided in a hind part of the wire rod part 42, and a second through hole 32a penetrating in a direction orthogonal to a long axis direction is provided in a forepart of the shaft part 32s. Then, the first through hole 42a and the second through hole 32a are disposed so as to overlap each other. A connecting member 84 such as a bolt is inserted into the shaft part 32s from outside so as to pass through the first through hole 42a and the second through hole 32a, and a tip of the connecting member 84 which is exposed outward from the shaft part 32s is fastened by a nut, so that the wire rod part 42 and the rotating body 32 are connected to each other.

The wire rod part 42 and the rotating body 32 can be connected to each other by the methods such as laser welding, solder welding, brazing, bonding, swaging, or screwing, for example, in the same method as the first embodiment.

The opening/closing operation part 62 and the tubular part 72 can be connected to each other in the same method as the opening/closing operation part 61 and the tubular part 71 according to the second embodiment. As shown in FIG. 11 and FIG. 12, for example, a third through hole 62a penetrating in a direction orthogonal to a long axis direction of the housing 21 and a hole part 62h by which at least a part of the tubular part 72 is housed in the opening/closing operation part 62 are provided in the opening/closing operation part 62. A hind part of the tubular part 72 is inserted into the hole part 62h of the opening/closing operation part 62, and connecting members 85 and 86 such as slotted set screws, for example, are inserted into the third through hole 62a. Then, the tubular part 72 is externally tightened, so that the opening/closing operation part 62 and the tubular part 72 are connected to each other.

The opening/closing operation part 62 and the tubular part 72 can be connected to each other by the methods such as laser welding, solder welding, brazing, bonding, swaging, or screwing, for example, in the same method as the second embodiment.

The clip part 52 is in a closed state where the clip part 52 is in the tubular part 72 when the opening/closing operation part 62 is moved to the clip part 52 side, and the clip part 52 is in an opened state where the clip part 52 comes out of the tubular part 72 when the opening/closing operation part 62 is moved to opposite to the clip part 52 side, in other words, is moved to a hind part of the housing 22. In the micro forceps 12 according to the present invention, a clipping direction of the clip part 52 can be aligned with an object through a rotation operation of the rotating body 32, so that there is no need to rotate the housing 22 itself around a long axis of the housing 22. Also, in the micro forceps 12 according to the present invention, the rotating body 32 and the tubular part 72 are not connected to each other, so that the tubular part 72 does not rotate even if the rotating body 32 is rotated, which allows less-invasive treatment. Further, in the micro forceps 12 according to the present invention, the clip part can be performed with the housing 22 being stably grasped. Thus, the micro forceps 12 can reduce a probability of occurrence of medical accidents such as displacement of a clipping position or misrecognition of a clipped object.

For the same reason as in the second embodiment, as shown in FIG. 12, the opening/closing operation part 62 preferably has a finger pad piece 66 projecting in a direction away from the long axis of the housing 22. Also, as with the second embodiment, in the cross section along the axis direction of the micro forceps 12, an outer end 66t of the finger pad piece 66 is preferably placed at an inner position of an outer end 27m of the opening part 27. Further, as with the second embodiment, it is preferred that the finger pad piece 66 is hidden from a view by the housing 22 in both of a case where the housing 22 is seen from a clip part 52 side in a long axis direction and a case where the housing 22 is seen from opposite to the clip part 52 side.

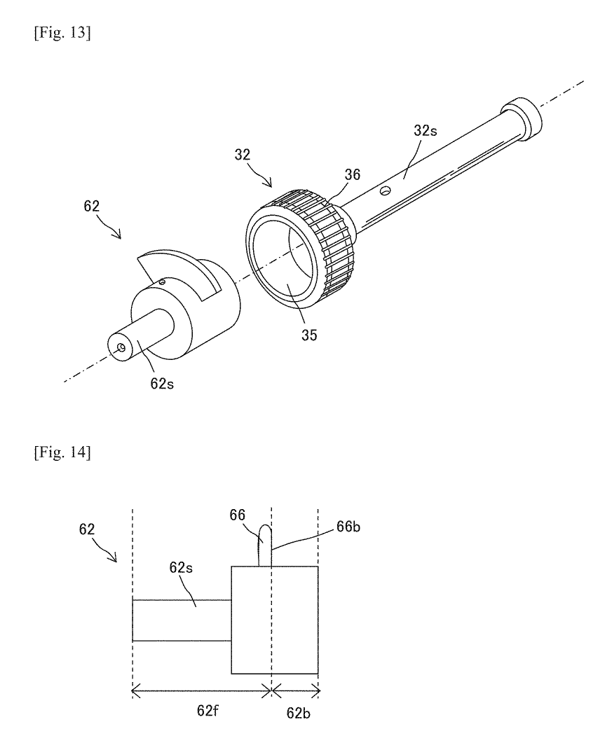

FIG. 13 is a perspective view showing an engagement between the rotating body 32 and the opening/closing operation part 62 according to the third embodiment. The rotating body 32 is provided with a projection part 36 on a periphery thereof and has a shaft part 32s. FIG. 14 is a side view of the opening/closing operation part 62 according to the third embodiment. The opening/closing operation part 62 can be divided into a front part 62f and a rear part 62b by the finger pad piece 66 as a boundary in a long axis direction of the housing 22. More specifically, as shown in FIG. 14, a boundary between the front part 62f and the rear part 62b of the opening/closing operation part 62 in a long axis direction of the housing 22 is set as a position of a rear end 66b of the finger pad piece 66. In a forepart of the front part 62f of the opening/closing operation part 62, a shaft part 62s connected to the tubular part 72 is provided.

It is preferred that a recessed part 35 is provided in the forepart of the rotating body 32, and the rotating body 32 is engaged with the opening/closing operation part 62 at the recessed part 35. That is, it is preferred that at least a part of the rear part 62b of the opening/closing operation part 62 is housed in the recessed part 35 of the rotating body 32 even if the opening/closing operation part 62 is positioned at a front end in a long axis direction of the housing 22 and the clip part 52 is into a closed state. The opening/closing operation part 62 is governed by the rotating body 32, so that displacement of the opening/closing operation part 62 and the rotating body 32 in a radial direction can be prevented. Also, even if the rotating body 32 is pressed inward in a radial direction, the rotating body 32 is not deformed and kept in the same state as it was before a press as far as the rear part 62b of the opening/closing operation part 62 is retained in the rotating body 32. Accordingly, it is possible to prevent a smooth rotating operation from being hampered due to entry of a whole of the rotating body 32 into the housing 22.

The number of opening parts 27 provided in the housing 22 is not limited to any particular number. For example, as shown in FIG. 9, in a case where the number of the opening parts 27 is one, both of the rotating body 32 and the opening/closing operation part 62 are exposed from the one opening part 27, so that the rotating body 32 and the opening/closing operation part 62 can be easily operated with one finger.

Also, though not shown in the drawings, in a case where two opening parts 27 are formed and the rotating body 32 and the opening/closing operation part 62 are exposed in one and the other of the two opening parts 27, respectively, the rotating body 32 and the opening/closing operation part 62 can be easily operated with different fingers, respectively.

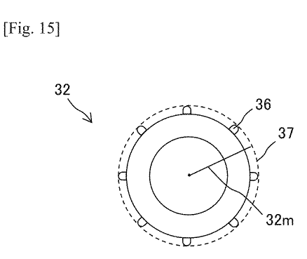

It is preferred that at least a part of the finger pad piece 66 of the opening/closing operation part 62 is positioned outer position of the largest radius 32m of the rotating body 32, in a radial direction. FIG. 15 is a front view of the rotating body 32 according to the third embodiment. In this regard, it is noted that the largest radius 32m of the rotating body 32 is a radius of a virtual circle 37 which is uniquely formed by at least three projection parts 36 provided on a periphery of the rotating body 32. By disposing the finger pad piece 66 of the opening/closing operation part 62 and the rotating body 32 in the above-described manner, it is possible to improve operability of the opening/closing operation part 62.

This application claims the benefit of the priority date of Japanese patent application No. 2014-217254 filed on Oct. 24, 2014. All of the contents of the Japanese patent application No. 2014-217254 filed on Oct. 24, 2014, are incorporated by reference herein.

### REFERENCE SIGNS LIST

10, 11, 12: a micro forceps
20, 21, 22: a housing
23: a side
25, 26, 27: an opening part
30, 32: a rotating body
35: a recessed part
40, 41, 42: a wire rod part
50, 51, 52: a clip part
60, 61, 62: an opening/closing operation part
65, 66: a finger pad piece
70, 71, 72: a tubular part
100: a tip part of a micro forceps
110, 111: an elastic member
120: a buffering member

1. A micro forceps comprising:
a housing having an opening part on a side thereof;
a rotating body that is retained in the housing, rotatable around a long axis of the housing, and partially exposed from the opening part;
a clip part for clipping an object;
a wire rod part provided with the clip part on one side, and connected to the rotating body on the other side,
an opening/closing operation part that is retained in the housing, movable along the long axis direction of the housing, and partially exposed from the opening part; and
a tubular part connected to the opening/closing operation part; wherein
the clip part is in a closed state where the clip part is placed in the tubular part when the opening/closing operation part is moved to a clip part side; and
the clip part is in an opened state where the clip part comes out of the tubular part when the opening/closing operation part is moved to opposite to the clip part side.

2. The micro forceps according to claim 1, wherein
the rotating body has a recessed part on the clip part side in the long axis direction of the housing and is engaged with the opening/closing operation part at the recessed part.

3. The micro forceps according to claim 1, wherein
an outer diameter of the tubular part is 1.1 mm or smaller.

4. The micro forceps according to claim 1, further comprising
a fixing means that fixes a rotating position of the rotating body.

5. The micro forceps according to claim 1, wherein
the wire rod part has a solid shape or a hollow shape in cross-section.

6. The micro forceps according to claim 1, wherein
the rotating body is engaged with the housing on an opposite side of the clip part side in the long axis direction of the housing.

7. The micro forceps according to claim 1, wherein
the housing and the opening/closing operation part are connected with each other by an elastic member.

8. The micro forceps according to claim 7, wherein
the elastic member is a coil spring.

9. The micro forceps according to claim 1, wherein
the opening/closing operation part has a finger pad piece projecting in a direction away from the long axis of the housing.

10. The micro forceps according to claim 9, wherein
an outer end of the finger pad piece is placed at an inner position of an outer end of the opening part.

11. The micro forceps according to claim 1, wherein
an opening range of the opening part in a circumferential direction of the housing is 60° or larger and 180° or smaller relative to the circumferential direction of the housing.

12. The micro forceps according to claim 1, wherein
the rotating body and the opening/closing operation part are exposed from the opening part.

13. The micro forceps according to claim 1, wherein
two of the opening part are formed, the rotating body is exposed from one of the opening part, and the opening/closing operation part is exposed from the other of the opening part.
